(19) United States
(12) Patent Application Publication
(10) Pub. No.: US 2019/0275082 A1
(43) Pub. Date: Sep. 12, 2019

(54) Scaffold Delivery of Immune Suppressors and Transplant Material for Control of Transplant Rejection

(71) Applicant: Northwestern University, Evanston, IL (US)

(72) Inventors: Xunrong Luo, Chicago, IL (US); Lonnie Shea, Chicago, IL (US); John G. Graham, Chicago, IL (US); Kathryn L. Pothoven, Chicago, IL (US); Xiaomin Zhang, Naperville, IL (US)

(21) Appl. No.: 16/178,267

(22) Filed: Nov. 1, 2018

Related U.S. Application Data
(63) Continuation of application No. 15/351,017, filed on Nov. 14, 2016, now abandoned.
Application No. 15/351,017 is a continuation of application No. 14/068,872, filed on Oct. 31, 2013, now abandoned.
(60) Provisional application No. 61/720,571, filed on Oct. 31, 2012.

(51) Int. Cl. A61K 35/17; A61L 27/54; A61L 27/58; A61L 27/56; A61K 35/39; A61L 27/38; A61L 27/18

(52) U.S. Cl. CPC A61K 35/17; A61L 27/54; A61L 27/58; A61L 2300/64; A61K 35/39; A61L 27/3804; A61L 27/18; A61L 27/56

(57) ABSTRACT

The present invention provides compositions, devices, and methods for the coordinated delivery of transplant material and immune suppressors for control of transplant rejection. In particular embodiments, immune suppression cells (e.g., regulatory T cells) and transplant material (e.g., cells, tissue, etc.) are provided within a delivery scaffold for transplant into a subject.

### STATEMENT REGARDING FEDERAL FUNDING

This invention was made with government support under Grant Nos. K08 DK070029 and R01 EB009919 awarded by National Institutes of Health. The government has certain rights in the invention.

### FIELD

The present invention provides systems, devices, and methods for the coordinated delivery of transplant material and immune suppressors for control of transplant rejection. In particular embodiments, immune suppression cells (e.g., regulatory T cells) and transplant material (e.g., cells, tissue, etc.) are provided within a delivery scaffold for transplant into a subject.

### BACKGROUND

Islet transplantation is the transplantation of isolated islets from a donor pancreas and into another person. It is an experimental treatment for type 1 diabetes mellitus. Once transplanted, the islets begin to produce insulin, actively regulating the level of glucose in the blood. Islets are usually infused into the patient's liver (Lakey J, Burridge P, Shapiro A (2003). “Technical aspects of islet preparation and transplantation”. Transpl Int 16 (9): 613-632). The patient's body, however, will treat the infused islets just as it would any other introduction of foreign tissue: the immune system will attack the islets as it would a viral infection, leading to the risk of transplant rejection. Thus, the patient needs to undergo treatment involving immunosuppressants, which reduce immune system activity.

Although beta-cell replacement via transplantation of allogeneic islets has been explored as a potential curative treatment for type 1 diabetes, clinical islet transplantation has thus far yielded disappointing results, with less than 10% of those transplanted remaining insulin independent after five years (see, e.g., Ryan E A, Paty B W, Senior P A, et al. Five-year follow-up after clinical islet transplantation. Diabetes 2005; 54 (7): 2060).

### SUMMARY

In some embodiments, the present invention provides systems comprising: (a) a delivery scaffold; (b) transplantable material; and (c) immune suppression cells. In some embodiments, the scaffold comprises a polymer matrix. In some embodiments, the scaffold is porous. In some embodiments, the polymer matrix comprises a biocompatible and biodegradable polymer. In some embodiments, the polymer matrix comprises poly(lactide-co-glycolide). In some embodiments, the scaffold is fabricated in any shape suitable for implantation into a transplantation site on a subject. In some embodiments, the scaffold comprises multiple layers. In some embodiments, the transplantable material comprises cells or a tissue. In some embodiments, the transplantable material comprises islet cells. In some embodiments, the immune suppression cells comprise Treg cells.

In some embodiments, the present invention provides methods for enhancing the incorporation of transplant material into a subject comprising: (a) providing the transplant material and immune suppression cells on or within a delivery scaffold; and (b) transplanting the delivery scaffold into a transplantation site on a subject. In some embodiments, the scaffold is porous. In some embodiments, the polymer matrix comprises a biocompatible and biodegradable polymer. In some embodiments, the polymer matrix comprises poly(lactide-co-glycolide). In some embodiments, the scaffold is fabricated in any shape suitable for implantation into a transplantation site on a subject. In some embodiments, the scaffold comprises multiple layers. In some embodiments, the transplantable material comprises cells or a tissue. In some embodiments, the transplantable material comprises islet cells. In some embodiments, the immune suppression cells comprise Treg cells. In some embodiments, the islet cells are transplanted in the subject to treat type 1 diabetes.

### BRIEF DESCRIPTION OF THE DRAWINGS

FIG. 1 shows graphs depicting islet graft survival on PLG scaffolds prolonged by Treg colocalization within the scaffold. (A) Blood glucose over time posttransplant of recipients of −Treg (red), +Treg in the scaffold (blue), and +Treg delivered intravenously (green). Each line represents an individual mouse. Dotted line indicates blood glucose measurement of 250 mg/dL. (B) Kaplan-Meyer survival of islet grafts over time. Two consecutive blood glucose measurements over 250 mg/dL was used to determine rejection.

FIG. 2 shows images depicting protection of PLG scaffold transplanted islets by Tregs is associated with robust insulin production and FoxP3+ Treg co-localization around islets. Grafts from days 7 (both −Treg and +Treg) and day 25 (−Treg) and day 33 (+Treg) were sectioned and stained for insulin and FoxP3. Left panels: graft sections from −Treg recipients; right panels: graft sections from +Treg recipients. Magnification 20×.

FIG. 3 shows immunofluorescent images of PLG scaffold transplanted islet grafts stained with CD11c and F4/80 antibodies. PLG scaffold transplanted islets are infiltrated with DCs and macrophages both −Tregs and +Tregs and primarily localize on the scaffold surface. Grafts from days 7 (both −Treg and +Treg) and day 25 (−Treg) and day 33 (+Treg) were sectioned and stained with indicated antibodies. Red: CD11c (left panels) or F4/80 (right panels); blue: Hoechst stain of nuclei. Dotted lines outline islets. Magnification 20×.

FIG. 4 shows immunofluorescent images of PLG scaffold transplanted islet grafts stained with CD4 and CD8 antibodies. PLG scaffold transplanted islets are infiltrated with CD4 and CD8 T lymphocytes that localize around islets in both −Treg and +Treg conditions. Grafts from days 7 (both −Treg and +Treg) and day 25 (−Treg) and day 33 (+Treg) were sectioned and stained with indicated antibodies. Red: CD4 (left) panels) or CD8 (right panels); blue: Hoechst stain of nuclei. Dashed circles outline individual islets. Dotted lines outline islets. Magnification 20×.

FIG. 5 shows protection of PLG scaffold transplanted islets by Tregs is not associated with peripheral upregulation of total Treg population. CD25+FoxP3+ Treg cells were assessed in the spleen and the graft draining lymph node (dLN) on day 25 (−Treg) and day 33 (+Treg). (A) Percentages of CD25+FoxP3+ Treg cells among all CD4+ cells in the spleen and dLN are shown in FACS plots. (B) Percentages of CD4+CD25+FoxP3+ cells among all live cells were used to estimate the total number of CD4+CD25+FoxP3+ cells in the spleen and dLN. Comparisons were made between −Treg and +Treg samples.

FIG. 6 shows PLG scaffold transplanted islets +Tregs prevent proliferation of BDC2.5 naïve CD4+ in vivo. (A) BDC2.5 naïve CD4+ cells were labeled with CFSE and injected intravenously into −Treg and +Treg transplant recipients. CFSE and anti-BDC clonotype stainings are shown on cells gated on size and CD4 from the dLN and spleen. The percentage of cells in the indicated gates is shown.

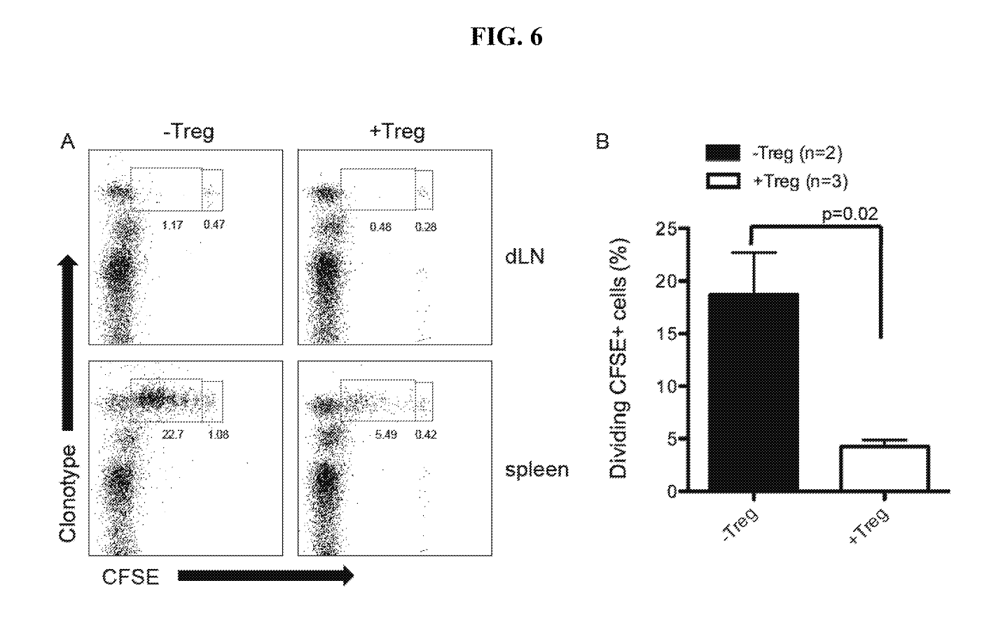

FIG. 7 shows PLG scaffold transplanted islets +Tregs induce systemic tolerance to islet grafts. (A) An additional islet transplant in the contralateral kidney capsule maintains euglycemia after PLG scaffold transplanted islets +Tregs is removed. Dotted line indicates blood glucose measurement of 250 mg/dL. (B) PLG scaffold islet graft at day 99 post-transplant with Treg co-localization around islets. (C) Second islet transplantation into contralateral kidney capsule is associated with robust Foxp3+ cell localization with islets.

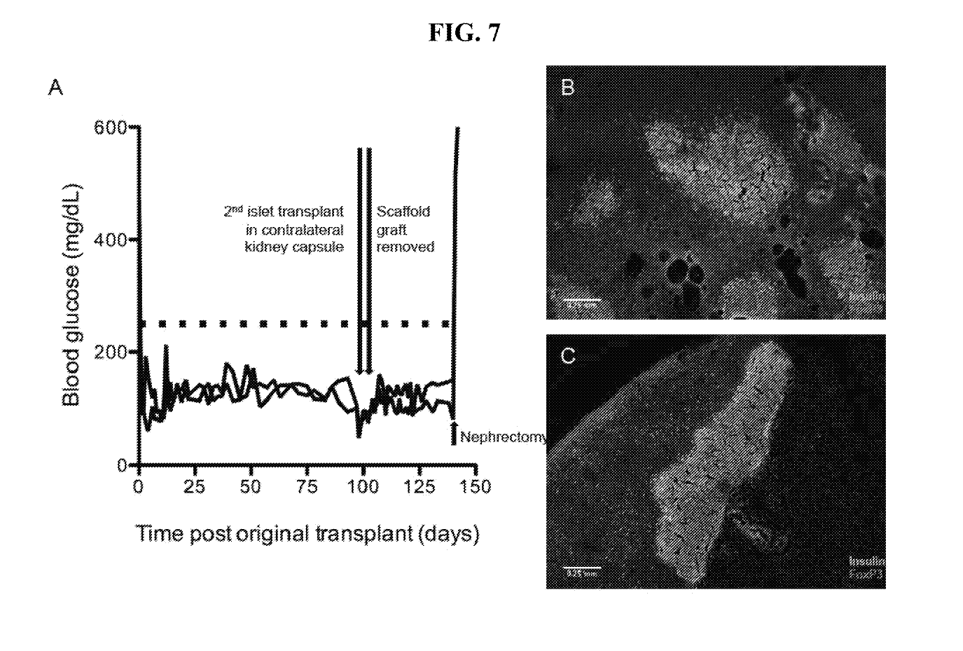

FIG. 8 shows islet grafts transplanted with BDC2.5 Vbeta4+ Tregs are infiltrated with Vbeta4-FoxP3+ Tregs over time. (A) Day 7 PLG scaffold transplanted islets with Vbeta4+FoxP3+ transplanted cells localizing around islets. (B) Day 33. (C) Day 99. (D). Day 43 post-transplant under the kidney capsule −Treg (original Treg post-transplant day 140). Dotted lines outline islets. White arrows indicated double positive Vbeta4+FoxP3+ cells. Magnification 20×.

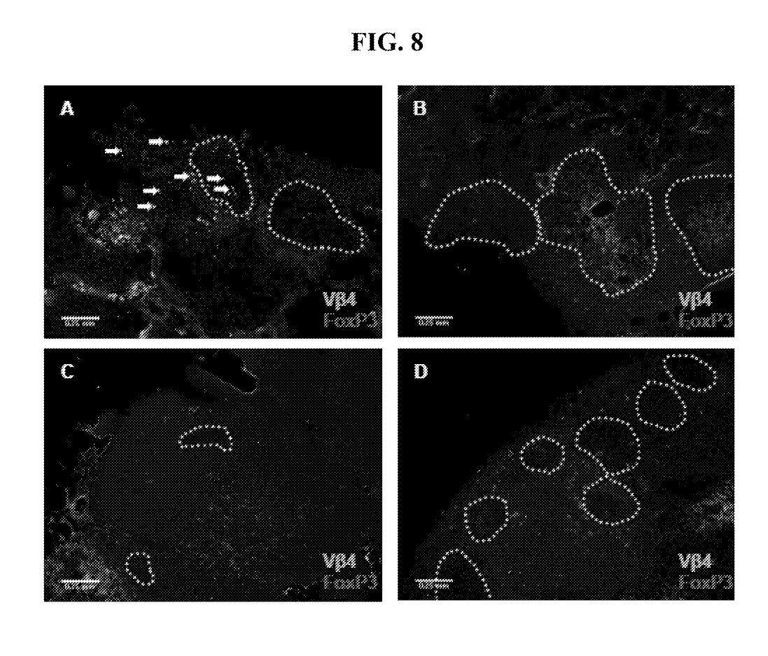

FIG. 9 shows a graph depicting the rate of diabetes appearance among+Treg and −Treg populations. NOD.scid recipients of splenocyte adoptive transfer from PLG scaffold transplanted islets. +Tregs became diabetic at a slower rate than PLG scaffold transplanted islets −Treg.

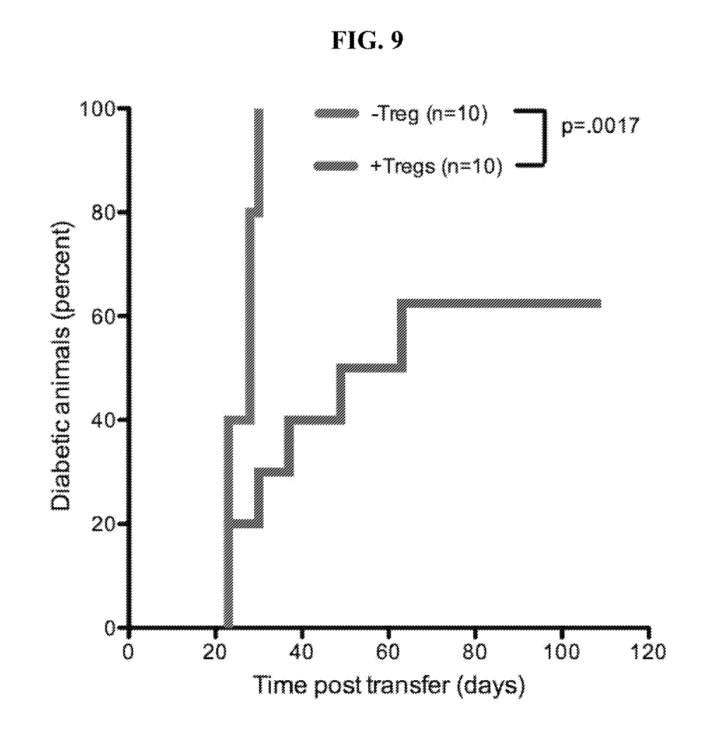

### DEFINITIONS

As used herein, the term “graft” refers to biological material derived from a donor for transplantation into a recipient. Grafts include such diverse material as, for example, isolated cells (e.g., islet cells), tissue, bone marrow, and organs. The graft is derived from any suitable source (e.g., mammalian, human, non-human primate, rodent, canine, porcine, feline, bovine, etc.), including human, whether from cadavers or living donors. The graft may be derived from the recipient (autograft), a genetically identical donor (isograft), a genetically distinct donor (allograft), or a donor of a different species (xenograft). The material may be taken directly from the donor and transferred to the recipient, or it may be cultured (e.g., in vitro) between extraction from the donor and transplant of the graft into/onto the recipient. As used herein, the term “transplant material” is synonymous with “graft.”

As used herein, the teim “donor” refers to the subject from which a graft is derived (e.g., directly or indirectly (in the case of cultured cells/tissues)). The donor may be of any suitable species (e.g., mammalian, human, non-human primate, rodent, canine, porcine, feline, bovine, etc.), and may be dead or alive upon extraction of the material (e.g., cells, tissue, etc.).

As used herein, the term “recipient” refers to the subject into/onto which a graft is transplanted. The recipient may be of any suitable species (e.g., mammalian, human, non-human primate, rodent, canine, porcine, feline, bovine, etc.).

As used here, the term “transplant” and variations thereof (e.g., transplanting, transplantation, etc.) refers to the transfer of a graft into/onto a recipient, whether the transplantation is syngeneic (where the donor and recipient are genetically identical), allogeneic (where the donor and recipient are of different genetic origins but of the same species), or xenogeneic (where the donor and recipient are from different species).

### DETAILED DESCRIPTION

In some embodiments, the present invention provides structures (e.g., scaffolds (e.g., polymer scaffolds (e.g., porous polymer scaffolds))) that serve as vehicles for delivering transplant material to specific sites within the body (See e.g., Lavik, E & Langer, R. Tissue engineering: current state and perspectives Appl Microbiol Biotechnol 65, 1-8 (2004); herein incorporated by reference in its entirety). Such scaffolds create synthetic microenvironments that, for example, promote new tissue formation (e.g., ingrowth) and reduce instances of transplant rejection (e.g., due to the scaffold, immune suppression agents, and/or other factors within the scaffold). Scaffolds may be configured to house one or more cell types for transplantation. In some embodiments, a scaffold is configured to house two or more cell types for transplantation (e.g., Tregs and islets). Some scaffolds are configured to contain and/or release one or more chemical and/or biological agents upon transplantation into a subject. Scaffolds may be of any suitable material and configuration, for example, the embodiments, highlighted below, and combinations thereof.

Although the specification specifically addresses transplantation of islet cells, the scaffolds and coordinated delivery methods described herein may find use in the transplantation of any suitably transplantable material (e.g., cells, tissues, organs, etc.). Scaffolds may be configured for specific delivery materials (e.g., tissue or cells) or may be capable of delivery of diverse materials (e.g., many different cell types, cell and tissues, etc.). Other suitable cell types for transplantation include, but are not limited to: stem cells, bone marrow cells, precursor cells (e.g., neural precursors), etc.

Similarly, although Tregs are specifically highlighted as immune suppressors, other cells or agents capable of producing a desired immunosuppressive effect may find use in embodiments of the present invention. Regulatory T cells (Treg), also known as suppressor T cells, are a subpopulation of T cells which downregulate the immune system, maintain tolerance to self-antigens, and downregulate autoimmune disease. In some embodiments, Tregs (e.g., CD4⁺, CD25⁺, and/or FoxP3⁺) provide tools for reducing autoimmunity and/or alloimmunity (e.g., to transplanted materials). Antigen-specific Tregs (e.g., CD4⁺, CD25⁺, and/or FoxP3⁺) control one or more immune responses including host autoimmunity and alloimmunity. Tregs may find use in, for example, controlling/reducing alloimmunity and/or recurrent autoimmunity in islet cell transplantation (e.g., autoimmunity which may impair long-term islet allograft function). Antigen-specific regulatory T cells can be generated in large numbers in vitro and adoptively transferred in vivo for protection of islet grafts. In addition, experiments conducted during development of embodiments of the present invention have shown that if co-transplanted with islets underneath the kidney capsule, these antigen-specific Tregs protect islet grafts from recurrent autoimmunity.

Scaffolds for islet transplantation in the treatment of Type 1 diabetes mellitus hold great promise for creating an alternative site of islet engraftment. The scaffold-created microenvironment may reduce the number of cells needed for successful treatment. Islet loss after transplantation is a major hurdle and has been attributed to several factors, including lack of ample blood supply, engraftment site, and the host immune response to foreign material. Scaffolds provide, for example, structural support (e.g., 2D or 3D support), a template for guiding engraftment, and for factor delivery (e.g., chemical, biological, or cellular agents). As few as 75 islets transplanted into the epididymal fat of mice on PLG scaffolds reverses streptozotocin-induced diabetes. While the scaffolds can support engraftment, in certain embodiments, a therapeutic strategy is employed to address the immune response. The present invention provides scaffold that can also serve as a vehicle for the delivery of transplant material (e.g., islet cells) as well as immune suppressors (e.g., regulatory T cells) to prevent rejection of the transplanted cells.

Experiments were conducted during development of embodiments of the present invention to investigate the co-delivery transplant material (e.g., tissues, cells (e.g., islet cells), etc.) and immune suppressors (e.g., immune suppression cells (e.g., regulatory T cells), etc.) at a clinically translatable transplant site using scaffold delivery (e.g., microporous scaffolds (e.g., PLG scaffolds)). Extrahepatic transplantation can avoid the instant blood-mediated inflammatory reaction and first-pass exposure to diabetogenic immunosuppression (Gibly and Graham; herein incorporated by reference in its entirety), while also presenting signals to promote engraftment (Salvay, 2008; herein incorporated by reference in its entirety). The NOD mouse model, which spontaneously develops autoimmune diabetes similar to human T1DM, was used to investigate the ability of antigen-specific Tregs to protect islet grafts from autoimmune destruction when cotransplanted with islets. Tregs were obtained by isolating and culturing T cells from a transgenic strain of NOD, NOD.BDC2.5 mice, which produce T cells that express only the T cell receptor against islet-antigen BDC. BDC2.5 naïve CD4+ T cells, in the presence of APCs, BDC peptide, and TGF-beta, differentiate and expand into CD4⁺CD25⁺Foxp3⁺Tregs in vitro. The mechanism of action of Tregs when colocalized with islet grafts was investigated by examining the ability of Tregs to induce infiltration, differentiation, and localization of other immune cell types into the graft and the phenotype and function of T cells in key locations for transplant tolerance including the dLN and spleen. Experiments were also to demonstrate local and/or systemic immunoprotection to islet antigens by locally delivered Tregs in islet transplantation.

Experiments were conducted during development of embodiments of the present invention to demonstrate the use of PLG scaffolds as a means to delivery islets and provide co-localization with Tregs (e.g., temporary or prolonged localization). In some embodiments, PLG scaffolds provide a localized space for islet transplantation that avoids issues associated with other transplantation techniques (e.g., at the hepatic site), including, but not limited to: the instant blood-mediated inflammatory response, a foreign extracellular matrix, and first-pass exposure to diabetogenic immunosuppression therapy, which combine to create a non-ideal transplant environment that may contribute to the loss of islets and insulin independence in both the short and long-term in the hepatic site. Furthermore, the microenvironment of the scaffold can be readily manipulated to enhance islet engraftment. In some embodiments, the porous scaffolds provided herein encourage cell infiltration and integration with the host, leading to revascularization of the transplanted islets. In some embodiments, PLG scaffolds provide an extra-hepatic and extra-renal transplant platform that allows for the co-localization of islets and Tregs while addressing the shortcomings of current clinical transplantation methods.

Experiments conducted during development of embodiments of the present invention demonstrated that Treg co-transplantation delayed or prevented rejection without systemic immunosuppression for delivery on a PLG scaffold into peritoneal fat. The scaffolds enable extra-hepatic, extra-renal transplantation, and these studies were conducted to demonstrate the efficacy of graft protection in setting of PLG scaffold transplanted islets by Tregs. PLG scaffold implantation leads to a foreign body response, a non-specific inflammatory response, which could complicate islet engraftment and strategies to promote immune protection. The process of implantation and the biomaterial recruits host APCs, such as macrophages and DCs, which can in turn induce secretion of inflammatory cytokines at the injury site. APCs did not localize to islet areas in either the Treg⁺ or Treg′ condition, and no significant differences in the presence or infiltrative patterns of these cells was observed in the scaffolds from control mice or mice treated with Tregs.

Graft protection by Tregs in PLG scaffold transplanted islet grafts is associated with both local and systemic regulatory mechanisms. At the graft level, a robust accumulation of FoxP3+ Tregs was observed that was localized around insulin-positive cells in the protected scaffold islet graft. The Tregs observed in the graft at day 7 were those initially transplanted (Vβ4+ BDC2.5 Tregs). However, at later time points, the Treg population that is localized around islet grafts shifts to FoxP3+Vβ4− Tregs. This result indicates that BDC2.5 Tregs induce and recruit host Tregs for long-term graft protection, most likely islet antigen-specific, including specificity for antigens causing autoimmune diabetes. BDC2.5 Tregs provide protection in a non-antigen-specific fashion against a diverse repertoire of autoreactive TCR specificities mediating diabetes in the NOD model. Transplanted Tregs localized in PLG scaffold islet grafts remain protective despite the plasticity of these cells in inflammatory environments and are able to protect islets from destruction by infiltrating CD4 and CD8 T cells.

Systemically, a second islet graft without Tregs implanted in a different location ˜100 days after the initial islet transplant with Tregs was protected from autoimmune destruction. Robust FoxP3+ cell infiltration was observed in the graft of this second transplant, indicating Treg trafficking to the site. In addition, the FoxP3+ cells localizing to the second islet graft were Vβ4-, indicating that host Tregs are induced by the initial BDC2.5 Treg transplantation and mediate long-term tolerance to islet grafts. However, adoptively transferred splenocytes from Treg⁺ mice induced diabetes, although at a slower rate than splenocytes from Treg⁻ controls. This induction of diabetes from splenocytes in tolerized mice indicates that autoreactive T cells remain in the host, yet the slower rate of diabetes onset suggests that these cells were present in smaller numbers or were possibly anergized by Treg contransplantation.

Experiments conducted during development of embodiments of the present invention demonstrate effective long-term protection of islet grafts from autoimmune destruction on PLG scaffolds when co-transplanted with antigen-specific Tregs. PLG scaffold transplanted islets with Tregs colocalized within the transplant site restore euglycemia and prolong islet graft survival, including permanent protection in a subset of recipients. Protection of these grafts is associated with Treg localization around islets. Initially, these Tregs are those transplanted at the time of islet transplantation, but recipient-derived Tregs replace the transplanted Tregs over time. This result indicates that islet antigen-specific Tregs induce tolerance to islet grafts through host-derived Tregs, likely islet antigen-specific as well. The infiltration by Tregs protected a second islet transplant, indicating a systemic tolerance to islet antigens. Nevertheless, autoreactive cells remain in the tolerized mouse although in reduced numbers or activity. In total, results from this study indicate a mixed localized and systemic mechanism of protection of islet grafts by Tregs when co-transplanted on PLG scaffolds.

The present invention provides compositions, devices, and methods for the coordinated delivery of transplant material and immune suppressors for control of transplant rejection. In particular embodiments, immune suppression cells (e.g., regulatory T cells) and transplant material (e.g., cells, tissue, etc.) are provided within a delivery scaffold for transplant into a subject. In some embodiments, the present invention provides co-transplantation of transplant material (e.g., cells (e.g., islets), tissue, etc.) and immune suppressors (e.g., immune suppression cells (e.g., regulatory T cells)) on and/or in a delivery scaffold (e.g., porous PLG scaffold).

In some embodiments, the present invention provides scaffolds for co-transplantation of graft material and immune suppressors. Scaffolds (e.g., PLG scaffolds) enable the co-localization of immune suppression agents (e.g., Tregs) and transplant material (e.g., islet graft) in a clinically suitable transplant site. In certain embodiments, containment within, or delivery upon, a scaffold reduces the amount of immune suppression agent (e.g., Tregs) necessary to ensure graft survival. Likewise, containment within, or delivery upon, a scaffold enhances the effectiveness of immune suppression agent (e.g., Tregs) thereby increasing graft survival.

In certain embodiments, the scaffolds provided herein are used for transplanting biological material (e.g., islet cells) and immunosuppressant agents (e.g., Tregs) into a subject for the treatment of diseases (e.g., type 1 diabetes), and related applications (e.g., diagnostic methods, research methods, drug screening).

Methods for fabricating porous poly(lactide-co-glycolide) (PLG) scaffolds have been previously described (Mooney, D. J., Baldwin, D. F., Suh, N. P., Vacanti, J. P. & Langer, R. Novel approach to fabricate porous sponges of poly(D,L-lactic-co-glycolic acid) without the use of organic solvents. Biomaterials 17, 1417-1422 (1996), herein incorporated by reference in its entirety; Harris, L. D., Kim, B. S. & Mooney, D. J. Open pore biodegradable matrices formed with gas foaming. J Biomed Mater Res 42, 396-402 (1998)), herein incorporated by reference in its entirety, and the ability to deliver proteins and DNA from such scaffolds documented (Richardson, T. P., Peters, M. C., Ennett, A. B. & Mooney, D. J. Polymeric system for dual growth factor delivery. Nat Biotechnol 19, 1029-1034 (2001), herein incorporated by reference in its entirety; Shea, L. D., Smiley, E., Bonadio, J. & Mooney, D. J. DNA delivery from polymer matrices for tissue engineering. Nat Biotechnol 17, 551-554 (1999), herein incorporated by reference in its entirety; Jang, J. H., Rives, C. B., & Shea, L. D. Plasmid delivery in vivo from porous tissue-engineering scaffolds: transgene expression and cellular transfection. Mo Therl 12, 475-483 (2005), herein incorporated by reference in its entirety; Sheridan, M. H., Shea, L. D., Peters, M. C. & Mooney, D. J. Bioabsorbable polymer scaffolds for tissue engineering capable of sustained growth factor delivery. J Control Release 64, 91-102 (2000)), herein incorporated by reference in its entirety. Certain limitations have been encountered with these scaffold technologies, namely the potential discrepancy involved in designing a scaffold with an optimal physical structure that simultaneously functions as an effective drug delivery device. In some instances, these two design considerations are not compatible, and it becomes a challenge to fabricate a scaffold that satisfies both design requirements. Accordingly, in some embodiments, the present invention provides a layered scaffold design to overcome such limitations. In some embodiments, the present invention provides a layered scaffold design having layers with different physical properties to serve different functions. Suitable methods of scaffold fabrication (as well as suitable scaffolds or portions thereof) are described in, for example, U.S. Pat. App. No. 2009/0238879; U.S. Pat. App. No. 2006/0002978; U.S. Pat. App. No. 2005/0090008; U.S. Pat. App. No. 2002/0045672; U.S. Pat. No. 7,427,602; herein incorporated by reference in their entireties.

In some embodiments, a scaffold is a substantially 2D surface (e.g., depth is less that 1% of its length and width). In other embodiments, a scaffold is a 3D matrix, platform, implant, particle, chip, etc. Scaffold may be of any suitable structural construction, including, but not limited to a slab of material (e.g., polymer), multiple layers (e.g., of polymer), a matrix, etc.

In some embodiments, a scaffold comprises one or more layers (e.g., 1, 2, 3, 4, 5, 6, 7, 8, 9, or more). In some embodiments, scaffold layers are configured for desired characteristics and/or performance of specific functions (e.g., biological/chemical agent release, tissue in-growth, containment of transplant material and/or immune suppressors, release of transplant material and/or immune suppressors, etc.). For example, an outer layer is highly porous to allow tissue in-growth and fluid/nutrient/cellular exchange while an inner layer is less porous and/or impermeable. Multiple layer scaffolds are described, for example, in U.S. Pat. App. No. 2009/0238879 which is herein incorporated by reference in its entirety.

A scaffold may comprise any combination and ordering of layers of differing compositions and characteristics (e.g., materials, density, porosity, permeability, etc.). For example, a scaffold may comprise a thin, non-porous center layer sandwiched between two highly porous outer layers. Such a configuration has been demonstrated (See U.S. Pat. App. No. 2009/0238879 which is herein incorporated by reference in its entirety) to exhibit enhanced capacity for delivery of, for example, pharmaceutical agents, DNA, RNA, and/or biological material (e.g., cells (e.g., pancreatic islet cells, Tregs, etc.). In other embodiments, one or more layers may comprise a chemical or biological agent is associated therewith (e.g., contained therein, adsorbed thereon, encapsulated therein, etc.).

In some embodiments, the scaffold is composed of one or more materials which are biodegradable and/or biorespobable. In some embodiments, the scaffold comprises one or more polymers. Suitable polymers include, for example, a polymer from the linear polyester family, such as polylactic acid, polyglycolic acid or polycaprolactone and their associated copolymers, e.g. poly(lactide-co-glycolide) at all lactide to glycolide ratios, and both L-lactide or D,L lactide. Polymers such as polyorthoester, polyanhydride, polydioxanone and polyhyroxybutyrate may also be employed. In some embodiments, a carrier comprises PLG. In other embodiments, the polymer matrix further comprises an aliphatic polyester, a polyanhydride, a polyphosphazine, a polyvinyl alcohol, a polypeptide, an alginate, or any combination thereof. In some embodiments, PLG polymer is composed of 50:50 D,L-lactide:glycolide, 65:35 D,L-lactide:glycolide, 75:25 D,L-lactide:glycolide, 85:15 D,L-lactide:glycolide, D,L-lactide alone, L-lactide alone, 25:75 D,L-lactide:ε-caprolactone, 80:20 D,L-lactide:ε-caprolactone, ε-caprolactone alone, or other suitable formulations (e.g., other ratios between 99:1 and 50:50, other polymer combinations, etc.). In certain embodiments, PLG polymers are terminated by a functional group of chemical moiety (e.g., ester-terminated, acid-terminated, etc.). In some embodiments, PLG is modified (e.g., with poly(ethylene glycol), with a functional group or chemical moiety). Different portions and/or layers of a scaffold may comprise different polymers or polymer ratios (e.g., to achieve desired characteristics (e.g., porosity, permeability, density, flexibility, etc.).

Scaffolds used in embodiments of the present invention may be made of any suitable materials (e.g., polymers) that are useful in chemical and/or biochemical synthesis. Such materials may include glasses, silicates, celluloses, synthetic resins, and polymers. Suitable polymers may include those listed in the preceding paragraph as well as others including, but not limited to: substantially pure carbon lattices (e.g., graphite), dextran, polysaccharides, polypeptides, polynucleotides, acrylate gels, polyanhydride, poly(lactide-co-glycolide), polytetraflouroethylene, polyhydroxyalkonates, cross-linked alginates, gelatin, collagen, cross-linked collagen, collagen derivatives, such as, succinylated collagen or methylated collagen, cross-linked hyaluronic acid, chitosan, chitosan derivatives, such as, methylpyrrolidone-chitosan, cellulose and cellulose derivatives such as cellulose acetate or carboxymethyl cellulose, dextran derivatives such carboxymethyl dextran, starch and derivatives of starch such as hydroxyethyl starch, other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyglycolide homoploymers, polyoxanones and polyoxalates, copolymer of poly(bis(p-carboxyphenoxy)propane)anhydride (PCPP) and sebacic acid, poly(1-glutamic acid), poly(d-glutamic acid), polyacrylic acid, poly(dl-glutamic acid), poly(1-aspartic acid), poly(d-aspartic acid), poly(dl-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), polybutylene diglycolate), nylon-2/nylon-6-copolyamides, polydihydropyrans, polyphosphazenes, poly(ortho ester), poly(cyano acrylates), polyvinylpyrrolidone, polyvinylalcohol, poly casein, keratin, myosin, and fibrin, silicone rubbers, or polyurethanes, and biocompatible and/or biodegradable derivatives and/or combinations thereof. The present invention also provides methods and assays to test materials and methods of preparation thereof for use in scaffolds described herein (See, for example, examples 1-4).

In some embodiments, a scaffold comprises one or more porous portions. In some embodiments, a scaffold is porous (e.g., microporous, mesoporous, macroporous, etc.). In some embodiments, a scaffold comprises one or more microporous segments, portions, and/or layers. Micropores have diameters of less than 2 nm. In some embodiments, a scaffold comprises one or more mesoporous segments, portions, and/or layers. Mesopores have diameters between 2 nm and 50 nm. In some embodiments, a scaffold comprises one or more macroporous segments, portions, and/or layers. Macropores have diameters of greater than 50 nm. In some embodiments, pore sizes of various segments, portions, and or layers of a scaffold are configured for the performance of specific functions (e.g., biological/chemical agent release, tissue in-growth, containment of transplant material and/or immune suppressors, release of transplant material and/or immune suppressors, etc.). A scaffold may comprise multiple layers, segments, portions, etc. with varying pore size (e.g., non-porous, microporous, mesoporous, macroporous, etc.).

In some embodiments, a scaffold comprises one or more impermeable segments, portions, and/or layers. In some embodiments, a scaffold comprises one or more permeable segments, portions, and/or layers. In some embodiments, scaffolds or portions thereof may be of any suitable permeability. In some embodiments, different regions/layers of a scaffold are configured to be permeable to different size, charge, and/or types of materials/objects. In some embodiments, the permeability of a region/layer of a scaffold is affected by the material composition (e.g., polymer make-up), degree of crosslinking, polymer modification, porosity, charge, etc. In some embodiments, a scaffold, or layer or portion thereof, is permeable to one or more cell types (e.g., islet cells, Tregs, etc.). In some embodiments, a scaffold, or layer or portion thereof, is permeable to macromolecular agents (e.g., polypeptides, proteins, lipids, nucleic acids, etc.), but not to most cell types (e.g., islet cells, Tregs, etc.). In some embodiments, a scaffold, or layer or portion thereof, is permeable to small molecules (e.g., organic molecules, H₂O, monomer units, etc.), but not to macromolecular agents (e.g., polypeptides, proteins, lipids, nucleic acids, etc.) or most cell types (e.g., islet cells, Tregs, etc.). In some embodiments, a scaffold combines regions/layers of varying permeability to produce a suitable/desirable platform for transplantations. In some embodiments, various segments, portions, and or layers of a scaffold comprise varying permeabilities configured for the performance of specific functions (e.g., biological/chemical agent release and/or retention, tissue in-growth, containment of transplant material and/or immune suppressors, release of transplant material and/or immune suppressors, etc.).

In some embodiments, the structure, composition, permeability, porosity, etc. of a scaffold, or a portion or layer thereof, is configured to promote tissue in-growth. In some embodiments, tissue in-growth promotes, for example, graft stabilization, graft integration, vascularization, tolerance, healing, etc. In some embodiments, the surface and/or surface exposed layer of a scaffold comprises suitable pore size for tissue ingrowth. In some embodiments, a scaffold comprises in-growth promoting agents (e.g., adsorbed to the scaffold, contained within pores, released from the scaffold, etc.).

All or a portion of the scaffold may be suitable for promotion of in-growth. In embodiments in which a scaffold comprises multiple layers, only a subset of the layers (e.g., outer layers, upper layers, etc.) may be amenable to and/or promote in-growth. Factors such as the permeability, porosity, material make-up, and additional aganets may all affect the degree to which a scaffold or portion (e.g., layer) thereof accept and/or promote tissue in-growth.

In some embodiments, a chemical or biological agent is associated (e.g., adsorbed onto, encapsulated within, etc.) a scaffold or a portion or layer thereof. In certain embodiments, agents are encapsulated in particles (e.g., microspheres, such as poly(lactide-co-glycolide) that comprise the scaffold. The present invention is not limited by the nature of the chemical or biological agents. Such agents include, but are not limited to, proteins, nucleic acid molecules, small molecule drugs, lipids, carbohydrates, cells, cell components, and the like. In some embodiments, two or more (e.g., 3, 4, 5, or more) different chemical or biological agents are included in the scaffold or a portion or layer thereof. Agents may be configured for specific release rates. For example, a first agent may release over a period of 30 days while a second agent releases over a longer period of time (e.g., 60 days, 70 days, 90 days, etc.). In some embodiments, one layer (e.g., inner layer) is configured for slow-release of a biological or chemical agent. Slow release provides release of biologically active amounts of the agent over a period of at least 30 days (e.g., 40 days, 50 days, 60 days, 70 days, 80 days, 90 days, 100 days, 180 days, etc.).

In some embodiments, scaffolds comprise one or more agents (e.g., chemical agents, biological agents, cells, etc.) adsorbed to the surface of the scaffold. In some embodiments, adsorbed agents enhance interactions with the cells and tissues of the transplant recipient. In some embodiments, adsorbed agents promote tissue in-growth, enhance islet function, reduce immune reaction, stabilize graft-recipient interaction, etc. Any suitable agents that enhance transplantation may be adsorbed to the scaffold surface. For example, extracellular matrix proteins (e.g., collagen IV, exendin-4, etc.) may be adsorbed to scaffolds enhance the function of transplanted islets.

In some embodiments, scaffolds comprise one or more agents (e.g., chemical agents, biological agents, cells, etc.) encapsulated or contained within the scaffold or a portion or layer thereof. In some embodiments, encapsulated and/or contained agents enhance interactions with the cells and tissues of the transplant recipient. Encapsulated and/or contained agents may promote tissue in-growth, enhance islet function, reduce immune reaction, stabilize graft-recipient interaction, etc. Any suitable agents that enhance transplantation may be encapsulated and/or contained within the scaffold. Agents may by encapsulated and/or contained within any portion of the scaffold. For example, agents may be contained within pores and/or encapsulated within pores that that are closed off from the surface of the scaffold. In embodiments in which scaffolds are produced from particle building blocks, agents may be encapsulated within and/or adsorbed to the particles, thereby building the agents into the scaffold upon its manufacture. Any suitable mechanism for containing agents within a scaffold may find use in certain embodiments.

In some embodiments, agents (e.g., chemical, biological, cellular, etc.) adsorbed to or encapsulated/contained within a scaffold are released into the surrounding environment (e.g., solution, tissue, fluid, etc.) over time (e.g., minutes, hours, days, weeks, months, or more). Conversely, agents may be retained within or upon the scaffold without such release.

### EXPERIMENTAL

### Example 1

### Tregs Prolong Islet Graft Survival when Colocalized on PLG Scaffolds

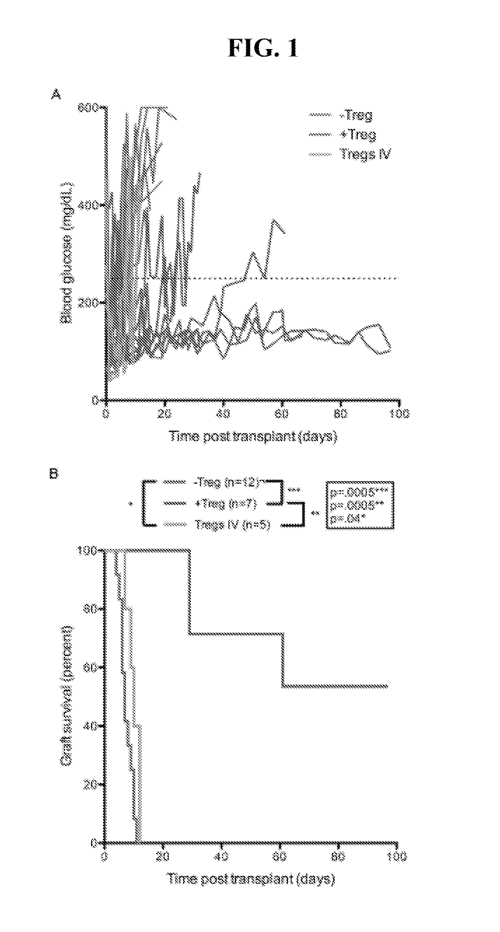

Experiments were conducted during development of embodiments of the present invention to investigate PLG scaffolds as a method to colocalize Tregs and islets in order to provide graft protection. Islets were transplanted on PLG scaffolds into the abdominal fat of diabetic female NOD recipients without Tregs (Treg⁻), with Tregs injected intravenously, and with Tregs localized in the PLG scaffold (Treg⁺s). The transplanted islets function in all conditions, indicated by normalized blood glucose levels compared to hyperglycemia observed in diabetic mice prior to transplantation. Without Treg cotransplantation, effector cells home to and target transplanted islet β-cells, resulting in graft destruction within 10 days of transplantation (SEE FIG. 1). In contrast to the Treg⁻ recipients, Treg⁺ PLG scaffold islet grafts delay or completely avoid rejection. However, when Tregs were delivered systemically, no protective effect was observed. These data demonstrate that Tregs can protect co-localized islets from autoimmune destruction with transplantation on PLG scaffolds into the abdominal fat.

### Example 2

### Histological Examination of PLG Scaffold Transplanted Islet Grafts with Tregs

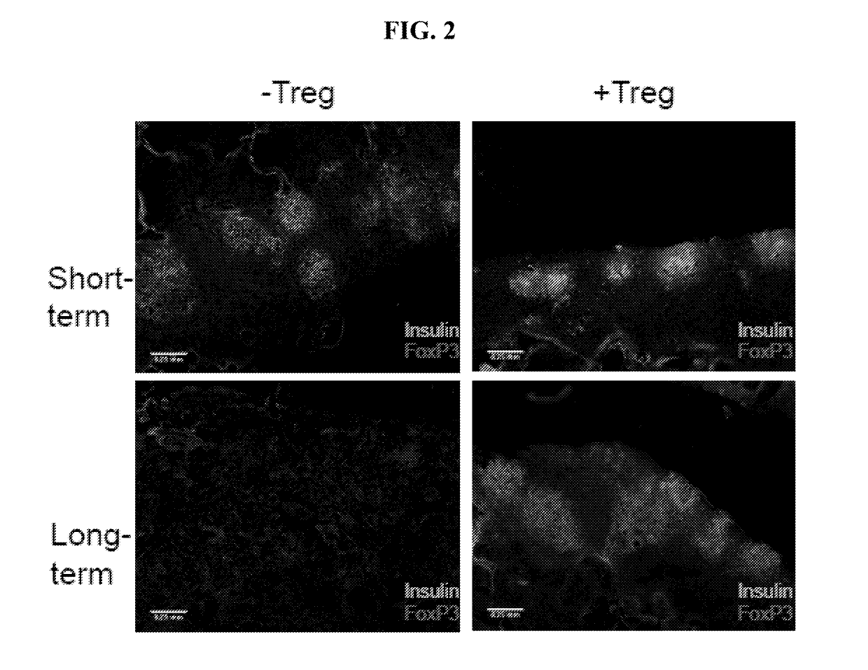

The distribution of islets and Tregs was examined by histological analysis in Treg+ and Treg⁻ islet transplant recipients. The PLG scaffolds were removed shortly before and after standard time for autoimmune-mediated rejection for both conditions, and day 99 post-transplant for Treg⁺. In Treg⁻ recipients, insulin-producing cells were detected at day 7 but were completely absent at day 25 (SEE FIG. 2). No FoxP3+ cells were observed within the scaffolds or near the islets before or after rejection. In contrast, for the Treg⁺ condition, insulin-producing cells were detected in short, intermediate, and long-term grafts, and the islet architecture was preserved at all Treg⁺ conditions. FoxP3+ cells were detected in close proximity to insulin-producing cells in all Treg⁺ recipients.

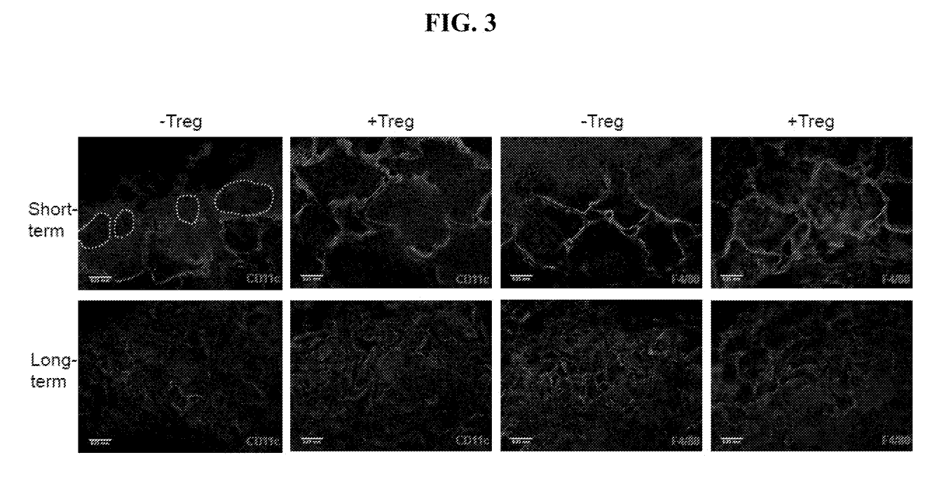

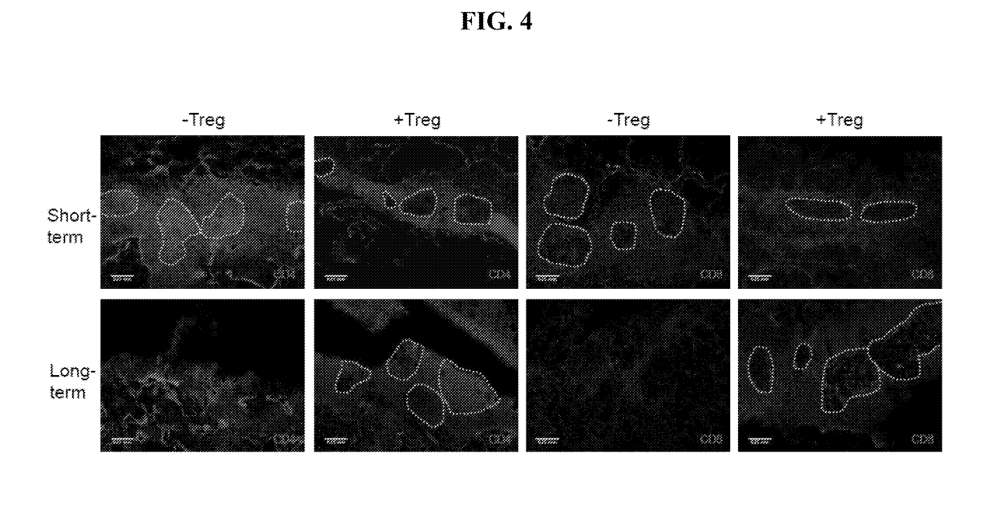

The grafts were examined for the presence and localization of additional immune cells important in protection and destruction of islet grafts. Anti-CD11c and F4/80 antibodies were used to identify dendritic cells (DCs) and macrophages respectively. F4/80 and CD11c staining was not observed around the islets in either Treg⁻ or Treg⁺ conditions (SEE FIGS. 3 and 4), but instead was observed to localize on the polymer surface of the PLG scaffold in both conditions. No differences were observed in infiltrating DCs or macrophages between grafts from Treg⁻ and Treg⁺. Over time, DCs and macrophages were observed on the polymer surface, consistent with the early time point. The number of CD11c+ and F4/80+ cells in the graft declines over time, and coincides with the degradation of the scaffold.

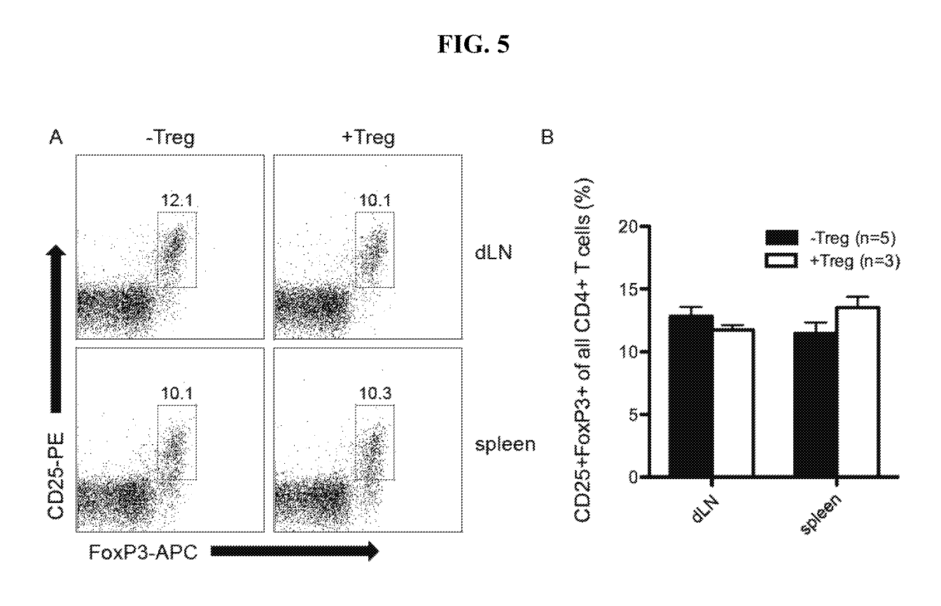

The presence of infiltrating CD4 and CD8 T cells was investigated, as they are normally responsible for rejection of transplanted islet grafts. Both CD4 and CD8 T cells infiltrated the scaffold and localized around islets before rejection in Treg⁻ recipients and both before and after standard rejection times in Treg⁺ recipients (SEE FIG. 5). In Treg⁺ recipients before and after typical rejection times, CD4 and CD8 T cells remain relatively peripheral to the islets. However, for day 7 Treg⁻ recipients, islet grafts are heavily infiltrated by CD4 cells, consistent with rejection, while CD8 T cells remain at the periphery of the islet.

### Example 3

### Treg Localization and Specificity

Experiments were conducted during development of embodiments of the present invention to characterize the distribution of Tregs. CD4+CD25+ FoxP3+ cells were quantified via FACS in the dLN of Treg⁻ and Treg⁺ mice, resulting in 12.8% and 11.7% of all CD4+ cells, respectively. CD4+CD25+FoxP3+ in the spleen of Treg⁻ and Treg⁺ were 11.4% and 13.5% of all CD4+ cells, respectively. The proportion of CD4+CD25+FoxP3− effector T cells in the dLN and spleen were not significantly different.

The suppressive function of T cells from the dLN and spleen of transplant recipients was investigated for the ability to suppress proliferation of islet-specific T cells in vitro. Splenic and dLN CD4+ T cells were isolated from post-rejection Treg⁻ and prolonged Treg⁺ PLG scaffold islet transplant recipients and treated as suppressor cells against naïve BDC2.5 CD4+ cells stimulated with SDC peptide and APCs. Proliferation counts were not significantly different compared to positive controls (no suppressive cells) in either CD4+ cells taken from the dLN or spleen between Treg⁻ and Treg⁺ islet transplant recipients. Therefore, CD4+ cells from the Treg⁺ dLN and spleen are not suppressive against islet-antigen-specific CD4 T cell proliferation in vitro, indicating that there is not an increased suppressive milieu in these locations because of Treg co-transplantation.

BDC2.5 Tregs were investigated for their ability to suppress islet-specific effector cells as a means of graft protection. BDC2.5 T cells proliferate significantly in the dLN, but not non-draining lymph nodes (ndLN). To verify the in vivo activity of BDC2.5 Tregs, naïve BDC2.5 CD4+ cells were labeled with carboxyfluorescein diacetate succinimidyl ester (CFSE) and transferred into Treg⁻ and Treg+ scaffold islet grafts recipients. BDC2.5 T cells were followed using a clonotype antibody specific for the BDC2.5 Vβ4 TCR. Few BDC clonotype+ cells were present in the ndLN and most had not diluted CFSE three days post-transplant in both Treg⁻ and Treg⁺ conditions. In contrast, in the dLN of Treg transplant recipients, clonotype+ T cells accumulated, 18.7% of total CD4+ T cells had undergone one or more divisions, as indicated by lower CFSE fluorescence. In the dLN of Treg⁺ transplant recipients, Vβ4+ T cells accumulated but the fraction of cells undergoing division was significantly lower at 4.27% of total CD4+ T cells, similar to ndLN. Therefore, BDC2.5 Tregs prevent the proliferation of islet-specific effector cells in vivo when transplanted with islets on PLG scaffolds.

### Example 4

### Systemic or Local Protection

Experiments were conducted during development of embodiments of the present invention to investigate whether Treg protection of islets was local or systemic. On day 97 post-transplantion of islets and Tregs on PLG scaffolds, a second islet graft was implanted into the contralateral kidney capsule without Tregs. On day 99, the scaffold originally transplanted with islets and Tregs was removed. Blood glucose was monitored for evidence of graft rejection for 43 days post-kidney capsule transplant, which corresponds to day 140 following the initial transplantation. The mice remained euglycemic until day 43 when a nephrectomy was performed, at which time the mice became hyperglycemic, indicating the kidney capsule graft was maintaining euglycemia. Histological staining of scaffold grafts (day 99) and kidney capsule grafts (day 140) for insulin and FoxP3 indicated that FoxP3+ cells were in close proximity to insulin-producing cells in scaffold grafts and both kidney capsules, indicating long-term immunoprotection of scaffold grafts and systemic protection of additional islet grafts without Tregs co-localized in a distant location.

The aforementioned scaffold and kidney capsule grafts were subsequently analyzed to determine the Treg source, whether they are derived from the originally transplanted Tregs or endogenously recruited. BDC2.5 Tregs exclusively express the Vβ4 T cell receptor; thus, all Tregs originally transplanted with islets on PLG scaffolds were FoxP3+Vbeta4+. Day 7, 33, and 99 Treg⁺ scaffold islet grafts and day 140 post-kidney capsule islet grafts were stained with FoxP3 and Vβ4 antibodies and examined for colocalization. FoxP3+Vbeta4+ cells were observed in the day 7 graft. However, day 33, 99, and a second kidney capsule contained predominantly FoxP3+Vβ4− cells, indicating that BDC2.5 Tregs induce other Treg infiltration and/or differentiation into PLG scaffold islet grafts. Vβ4+ Tregs were not observed in islet grafts after day 7, indicating that BDC2.5 Treg mechanism of action does not require their persistent presence in the transplant recipient for long-term graft protection; however, the present invention is not limited to any particular mechanism of action and an understanding of the mechanism of action is not necessary to practice the present invention.

Experiments were conducted during development of embodiments of the present invention to determine if autoreactive cells remained present in mice that were transplanted with islets and Tregs or if systemic anergy or deletion occurred with Treg transplantation. Splenocytes were isolated from Treg⁻ and Treg⁺ PLG scaffold islet graft recipients post-transplant day 33 and adoptively transferred via intravenous injection into normoglycemic male NOD.scid mice, which would not normally become diabetic. All mice that received splenocytes from Treg⁻ recipients became diabetic by day 29 while diabetes induction is significantly delayed with splenocytes from Treg⁺ donors, including 40% of the recipients from the Treg⁺ donor group that remained euglycemic for the duration of the study. These results indicate that autoreactive cells were present in mice that had protected islet grafts by Tregs, but the autoreactive cells were lower in number or were made anergic by Treg co-transplantation.

What is claimed is:

1. A system comprising:
(a) a delivery scaffold;
(b) transplantable material; and
(c) immune suppression cells.

2. The system of claim 1, wherein the scaffold comprises a polymer matrix.

3. The system of claim 1, wherein the scaffold is porous.

4. The system of claim 2, wherein the polymer matrix comprises a biocompatible and biodegradable polymer.

5. The system of claim 4, wherein the polymer matrix comprises poly(lactide-co-glycolide).

7. The system of claim 1, wherein the scaffold is fabricated in any shape suitable for implantation into a transplantation site on a subject.

8. The system of claim 1, wherein the scaffold comprises multiple layers.

9. The system of claim 1, wherein the transplantable material comprises cells or a tissue.

10. The system of claim 9, wherein the transplantable material comprises islet cells.

11. The system of claim 1, wherein the immune suppression cells comprise Treg cells.

12. A method for enhancing the incorporation of transplant material into a subject comprising:
(a) providing the transplant material and immune suppression cells on or within a delivery scaffold; and
(b) transplanting the delivery scaffold into a transplantation site on a subject.

13. The method of claim 12, wherein the scaffold comprises a polymer matrix.

14. The method of claim 12, wherein the scaffold is porous.

15. The method of claim 13, wherein the polymer matrix comprises a biocompatible and biodegradable polymer.

16. The method of claim 13, wherein the polymer matrix comprises poly(lactide-co-glycolide).

17. The method of claim 12, wherein the transplantable material comprises cells or a tissue.

18. The method of claim 17, wherein the transplantable material comprises islet cells.

19. The method of claim 12, wherein the immune suppression cells comprise Treg cells.

20. The method of claim 18, wherein the islet cells are transplanted in the subject to treat type 1 diabetes.
